(19) United States
(12) Patent Application Publication
(10) Pub. No.: US 2015/0165136 A1
(43) Pub. Date: Jun. 18, 2015

(54) Medicament Delivery System Having a Selectable Delivery Spray Pattern and a Method of its Use

(71) Applicant: STELA Medical, LLC, Waunakee, WI (US)

(72) Inventors: Richard Edward Galgon, Waunakee, WI (US); Katherine Lorraine Baldwin, Apple Valley, MN (US); James French Dorrance, Rice Lake, WI (US); Terah Michelle Hennick, Sun Prairie, WI (US); Alyssa Ruth Mitchell, Menasha, WI (US); Mitchell Eugene Tyler, Madison, WI (US)

(21) Appl. No.: 14/566,173

(22) Filed: Dec. 10, 2014

Related U.S. Application Data
(60) Provisional application No. 61/915,299, filed on Dec. 12, 2013.

(51) Int. Cl. A61M 11/00; A61M 19/00

(52) U.S. Cl. CPC A61M 11/00; A61M 19/00; A61M 2210/1035; A61M 2210/1032; A61M 2210/1028; A61M 2210/0625; A61M 2210/0618; A61M 2210/065

(57) ABSTRACT

Provided herein are medicament delivery systems for selectively providing radial, distal, or a combination radial and distal delivery spray patterns of a medicament, and a method of their use to treat a patient's airway. Generally, a medicament delivery system comprises an elongate shaft, a medicament source, and a means for controlling the delivery spray pattern of the elongate shaft. In use, the medicament delivery system is inserted into a patient's airway during a laryngoscopy procedure to deliver a topical anesthetic to the airway. The medicament delivery device can be used with both direct and indirect view laryngoscopy procedures.

### PRIORITY

The present application claims priority to U.S. provisional application Ser. No. 61/915,299 filed on 12 Dec. 2013, the entire content of which is incorporated by reference herein.

### FIELD

The present disclosure relates to a medicament delivery system having a selectable delivery spray pattern and a method of its use, for example during either direct or indirect view laryngoscopy procedures.

### BACKGROUND

Laryngoscopy is a procedure performed to obtain a view of the vocal fold and sub-glottal region. Laryngoscopes are inserted to lift anatomical structures that would normally be obstructing the vocal fold region and are performed for many reasons including patient throat pain or vocal fold analysis. Another common use of a laryngoscope is to assist with placement of an endotracheal (“tracheal”) tube during procedures requiring a patient to be placed under general anesthesia. While under general anesthesia, a tracheal tube is vital to maintain and control patient breathing through the procedure and prevent airway soiling.

Approximately 60,000 patients in the United States undergo general anesthesia every day (nearly 22 million each year). In approximately two thirds of these cases (14.7 million annually), an endotracheal tube is placed for airway protection and maintenance. During laryngoscopy, anesthesiologists now have the option to use one of two general types of laryngoscopes: direct or indirect. Direct laryngoscopes are rigid devices that allow the physician to use direct line of vision to observe the vocal fold region of a patient's airway with the purpose of inspection and intubation. This is the most conventional way to perform laryngoscopy. However, difficult airways, in which a direct line of sight cannot be established, have become more prevalent due to the recent rise in obesity. An increase in soft palate tissue occludes the patient's airway and anesthesiologists after find it more effective to perform indirect laryngoscopy that instead utilizes either optic or video means to view to patient's vocal fold region.

However, regardless of which laryngoscopy procedure is used to visualize a patient's airway, the presence of the tracheal tube, and/or the laryngoscope commonly results in adverse patient side effects including hypertension, tachycardia, tachyarrhythmia, increased intracranial and intraocular pressures, coughing, and sore throat if untreated. Topical application of local anesthetic (e.g. lidocaine) to the larynx and trachea prior to intubation and at the time of awakening from general anesthesia and extubation reduces the incidence and severity of these events.

Applicators currently on the market do not meet all the needs of a topical anesthetic applicator for use in both direct and indirect view laryngoscopy. Such applicators are currently designed for use only with direct view laryngoscopes. The features that make these current applicators suitable for use with direct view laryngoscopes would typically make the same applicator unsuitable for use with indirect view laryngoscopes. For example, applicators currently on the market either have rigid properties preventing the applicator from easily traversing the curvature of the pharynx without fracture, or on the other hand, are highly malleable, resulting in contortion upon contact with anatomical structures and thus preventing placement of the applicator. Furthermore, the devices currently on the market are not as effective as possible in delivering topical anesthetic to a patient's airway during laryngoscopy. For example, current applicators are shorter than the average tracheal tube and cannot be threaded through a tracheal tube, preventing their use with laryngoscopes preloaded with a tracheal tube or in anticipation of patient awakening and extubation. For these reasons, there is a clinical need for a universal endotracheal anesthetic applicator that overcomes the limitations of current devices and enables effective anesthetic application during all types of laryngoscopy.

### SUMMARY

In some embodiments, the medicament delivery system comprises an improved ability to selectively deliver medicament. Specifically, in some embodiments, the medicament delivery system provides enhanced delivery of a topical anesthetic to a patient's airway during both direct and indirect view laryngoscopy procedures.

In some embodiments, the medicament delivery system generally comprises an elongate shaft connected to a medicament source, and a component for controlling the delivery spray pattern of the elongate shaft. The medicament delivery system allows a user to select between a distal only, radial only, or combination radial and distal delivery spray pattern. Such delivery spray pattern selectivity allows the present medicament delivery system to deliver medicament in a more efficient manner. For example, the combination radial and distal delivery spray pattern allows for increased area coverage of medicament delivery. In situations where the elongate shaft is disposed inside another tubular member (such as, for example inside a tracheal tube) the distal only delivery spray pattern allows for more efficient use of the medicament by limiting the amount of medicament that would otherwise be lost inside the tubular member.

In addition to allowing a user to choose between a distal only, a radial only, or a combination radial and distal delivery spray pattern, the mechanism for controlling the delivery spray pattern of the elongate shaft may further comprise a component for more precisely controlling the radial circumference of the delivery spray pattern. For example, where the component for controlling the delivery spray pattern comprises a stopcock, the bore of the stopcock may be provided with any number and configuration of channels that allow a user to selectively choose among delivery spray patterns in which the radial delivery spray pattern is, for example, limited to a top half, bottom half, upper right quadrant, left side, etc. of the longitudinal length of the elongate shaft.

In some embodiments, the elongate shaft also incorporates any number of features for increasing the steerability of the device, such as: a preformed distal curve, a stylet, a braided or woven portion, etc. The enhanced steerability of the elongate shaft allows the elongate shaft to easily be positioned in a patient's airway in either direct view or indirect view laryngoscopy procedures without the elongate shaft contorting or fracturing. Furthermore, the longer length of the elongate shaft allows the elongate shaft to be preloaded into a tracheal tube prior to placement in the airway, thus decreasing the number of instruments and number of times instruments are inserted into a patient during a laryngoscopy procedure.

Thus, the enhanced features of the medicament delivery system allow the system to be used with both direct view and indirect view (including both channeled and non-channeled indirect view) laryngoscopes. The universal applicability of this system therefore allows hospitals to stock only one type of device for all laryngoscopy procedures to be performed, as the medicament delivery system of the device is compatible with all types of laryngoscopes and has superior delivery spray pattern and handling characteristics.

In an embodiment, the medicament delivery system of the present disclosure comprises a multi-lumen elongate shaft having first and second lumens. A plurality of outlet ports are provided along the outer surface of the elongate shaft. The plurality of outlet ports are fluidly connected to the first lumen of the elongate shaft so as to provide a radial delivery spray pattern. The distal end of the second lumen is provided with a distal outlet that provides a distal delivery spray pattern. In a further embodiment, the distal ends of the first and second lumens may be closed off by a cap having a distal outlet. Additionally, the medicament delivery system comprises a medicament source comprising a medicament.

The medicament delivery system further comprises a component for controlling the delivery spray pattern so as to allow a user to select a radial delivery spray pattern, a distal delivery spray pattern, or a combination of a radial and distal delivery spray pattern. In an embodiment, the component for controlling the delivery spray pattern of the device comprises a stopcock comprising a casing and a bore having two or more channels extending therethrough. At least one of the two or more channels of the bore has a cross-section equal to the cross-section of the elongate shaft first lumen, wherein at least one of the other two or more channels has a cross-section equal to the cross-section of the elongate shaft second lumen. The bore may also comprise a third channel having a cross-section equal to the cross-section of the first elongate shaft lumen. A portion of the third channel cross-section is blocked off by a protrusion extending into the third channel.

In an embodiment, a method of providing a medicament to a patient's airway comprises the steps of inserting and positioning an elongate shaft that selectively provides a radial delivery spray pattern, a distal delivery spray pattern and a combination radial and distal delivery spray pattern into a patient's airway. A user selects between a radial delivery spray pattern, a distal delivery spray pattern, or a combination of a radial and a distal delivery spray pattern and applies a medicament to the patient's airway via the elongate shaft. After selecting and applying medicament via a first delivery spray pattern, the user may subsequently select a second, different delivery spray pattern to deliver the same or a different medicament.

Additionally, the method may comprise the step of inserting and positioning a laryngoscope and a tracheal tube into the patient. In an embodiment, the elongate shaft is placed inside the tracheal tube prior to insertion of the tracheal tube into the patient. The combination elongate shaft/tracheal tube is then advanced into the patient's airway simultaneously. After applying the medicament, the elongate tube may be removed from the patient. In a further embodiment, the elongate tube may be reintroduced into the patient by inserting the elongate tube through the tracheal tube. After reintroducing the elongate tube a medicament maybe be applied via a user selected delivery spray pattern. The elongate tube and the tracheal tube may then both be removed from the patient.

In an embodiment, a medicament delivery system is provided and includes a medicament source, a flexible elongate shaft having a distal end and a proximal end. A radial delivery lumen and a distal delivery lumen extend along the length of the elongate shaft. A distal outlet is fluidly connected to the distal end of the elongate shaft. A plurality of ports are spaced radially along the length of the elongate shaft. The ports are fluidly connected to the radial lumen. The medicament delivery system includes a stopcock. The stopcock includes a casing and a rotatable bore in the casing. The casing includes an inlet stem attached to the medicament source and an outlet stem attached to the proximate end of the elongate shaft. The bore includes a first channel and a second channel. The first channel has a cross-section that matches the cross-section of the radial delivery lumen and the distal delivery lumen. The second channel has a cross-section that matches the cross-section of the distal delivery lumen. Rotation of the bore to a first position places the first channel in fluid communication with (i) the radial delivery lumen and the distal delivery lumen and (ii) the medicament source. In the first position, the elongate shaft delivers a radial delivery pattern and a distal delivery spray pattern. Rotation of the bore to a second position places the second channel in fluid communication with (i) the distal delivery lumen and (ii) the medicament source. In the second position, the elongate shaft delivers a distal delivery spray pattern.

### BRIEF DESCRIPTION OF THE DRAWINGS

FIG. 1A is a side perspective view of a medicament delivery system set to deliver a combination radial and distal delivery spray pattern in accordance with an embodiment of the present disclosure.

FIG. 1B is a side perspective view of the medicament delivery system of FIG. 1A set to deliver a distal only delivery spray pattern.

FIG. 2 is a side perspective view of a medicament delivery system connected to a medicament source in accordance with an embodiment of the present disclosure.

FIG. 3A is a side perspective view of a cap defining a distal outlet in accordance with an embodiment of the present disclosure.

FIG. 3B is a side perspective view of a nozzle defining a distal outlet in accordance with an embodiment of the present disclosure.

FIG. 4A is a cross-section of the elongate shaft in accordance with an embodiment of the present disclosure.

FIG. 4B is a cross-section of the elongate shaft in accordance with another embodiment of the present disclosure.

FIG. 5 is a cross-section of the elongate shaft in accordance with various other alternative embodiments of the present disclosure.

FIG. 6 is a magnified perspective view of a stopcock device in accordance with an embodiment of the present disclosure.

FIG. 7 is a cross-section of a stopcock having a threaded bore/casing connection in accordance with an embodiment of the present disclosure.

FIG. 8A is a side perspective view of a stopcock bore having a ridge connection in accordance with an embodiment of the present disclosure.

FIG. 8B is a side perspective view of a stopcock bore having a pillar connection in accordance with an embodiment of the present disclosure.

FIG. 8C is a side perspective view of a stopcock casing having a track capable of being used with the stopcock bore of FIG. 8A in accordance with an embodiment of the present disclosure.

FIG. 9A is a side perspective view of a casing outlet usable with an elongate shaft having a cross-section as shown in FIG. 4A in accordance with an embodiment of the present disclosure.

FIG. 9B is a side perspective view of a casing outlet usable with an elongate shaft having a cross-section as shown in FIG. 4A in accordance with another embodiment of the present disclosure.

FIG. 9C is a side perspective view of a casing outlet usable with an elongate shaft having a cross-section as shown in FIG. 4B in accordance with an embodiment of the present disclosure.

FIG. 9D is a side perspective view of a casing outlet where the elongate shaft is connected to the stopcock by positioning the elongate shaft inside the casing outlet in accordance with an embodiment of the present disclosure.

FIGS. 10A-H illustrate various configurations of additional channels of the stopcock bore in accordance with various other alternative embodiments of the present disclosure.

### DEFINITIONS

In describing and claiming the present disclosure, the following terminology will be used in accordance with the definitions set forth below.

The singular forms “a,” “an,” and, “the” include plural referents unless the context clearly dictates otherwise. Thus, for example, reference to “a lumen” includes reference to one or more of such lumens, and reference to “the attachment” includes reference to one or more of such attachments.

As used herein, the term “about” is used to provide flexibility to a numerical range endpoint by providing that a given value may be “a little above” or “a little below” the endpoint.

As used herein, the term “substantially” refers to the complete or nearly complete extent or degree of an action, characteristic, property, state, structure, item, or result. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

As used herein, “delivery” will be understood to comprise any type of delivery including, but not limited to: spraying, misting, flowing, infusing, injecting (via e.g., a high velocity flow), smearing, running, oozing, seeping, etc.

### DETAILED DESCRIPTION

The following description provides illustrative embodiments of the disclosure to highlight certain features. It should be understood that the disclosure is not limited to these particular embodiments.

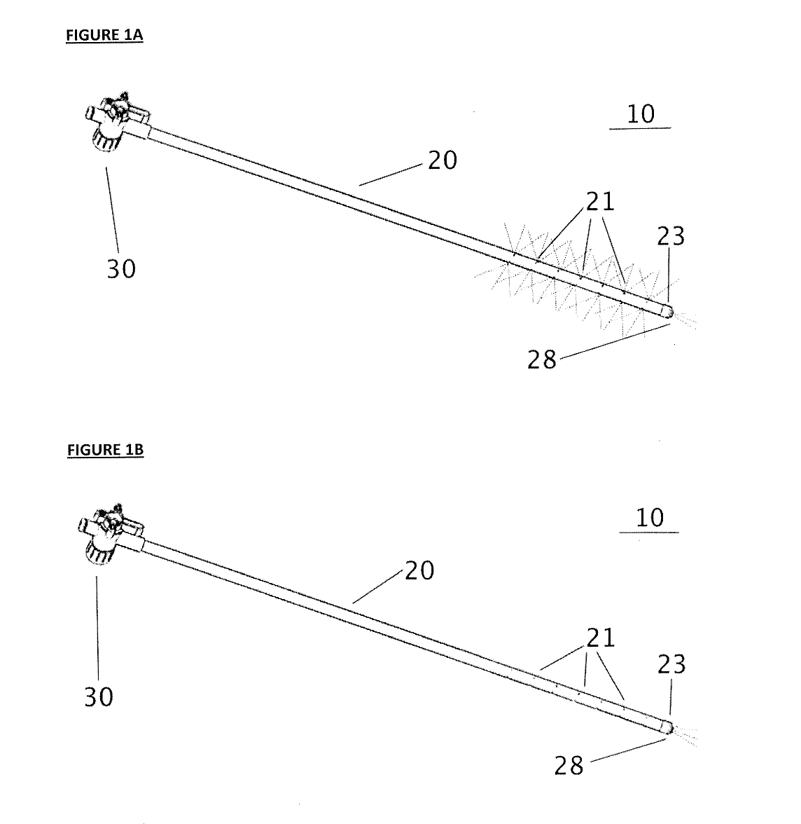

In an embodiment, the present disclosure provides a medicament delivery system 10 (FIGS. 1A and 1B) and associated method of its use.

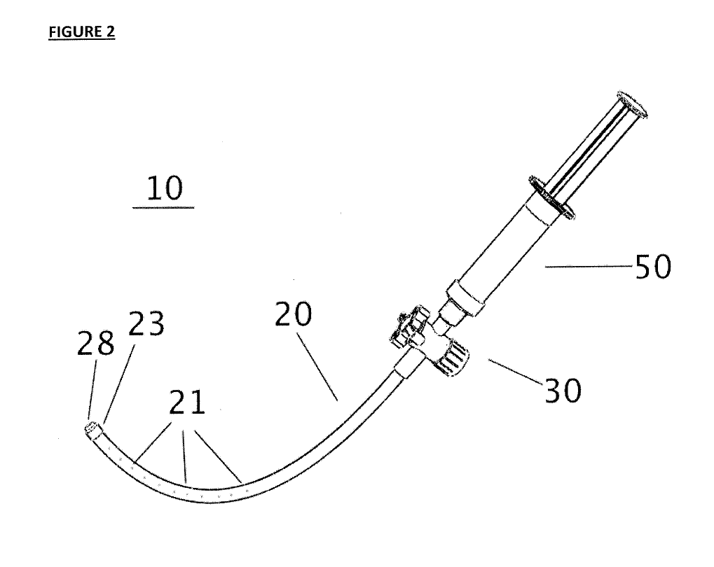

With respect to FIGS. 1A, 1B, and 2, a medicament delivery system 10 according to an embodiment of the present disclosure is illustrated. The medicament delivery system 10 comprises an elongate shaft 20, a component for controlling the delivery spray pattern of the elongate shaft 20 (hereafter a mechanism), and a medicament source. The length of the elongate shaft 20 may be varied depending on the specific requirements of the patient being treated and or the specific requirements of the method the elongate shaft 20 is to be used in. In an embodiment, the length of the elongate shaft 20 is from 20 cm, or 25 cm to 30 cm, or 35 cm, or 40 cm.

As seen in FIGS. 1A, 1B, and 2, the outer surface of the elongate shaft 20 is provided with a plurality of outlet ports 21. As seen in FIG. 1A, outlet ports 21 provided on the outer surface of the elongate shaft 20 allow the medicament delivery system 10 to achieve a radial delivery spray pattern. The radial delivery spray pattern may comprise a delivery spray pattern extending around the entirety of the circumference of the elongate shaft 20. Alternatively, the radial delivery spray pattern may be limited to only a portion of the circumference of the elongate shaft 20 (e.g. radial delivery extending only along a bottom portion of the longitudinal axis of the elongate shaft 20, etc.). The distal end of elongate shaft 20 is also provided with a distal outlet 28. As seen in FIGS. 1A and 1B, distal outlet 28 at the distal end of the elongate shaft 20 allows the medicament delivery system 10 to achieve a distal delivery spray pattern.

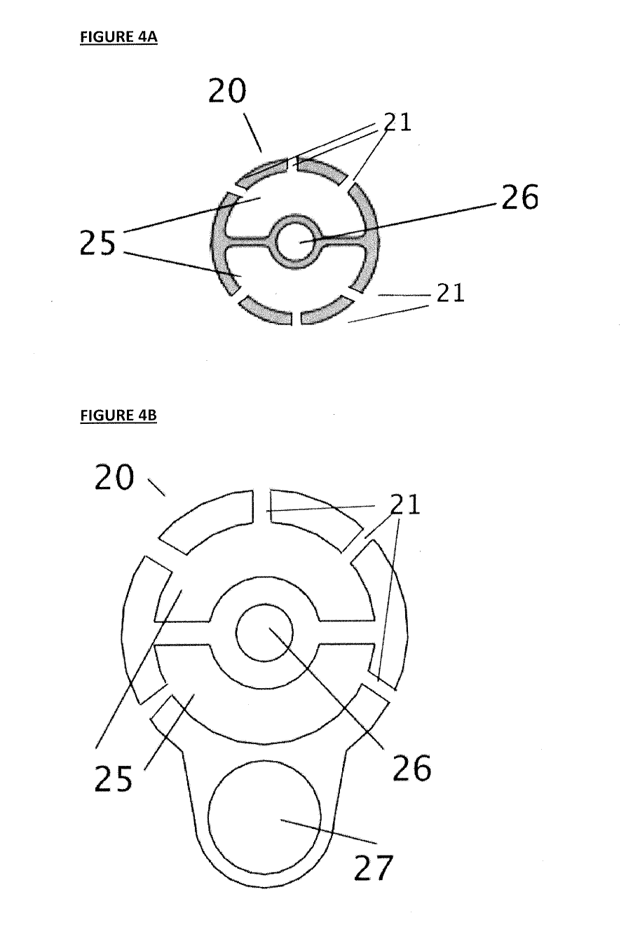

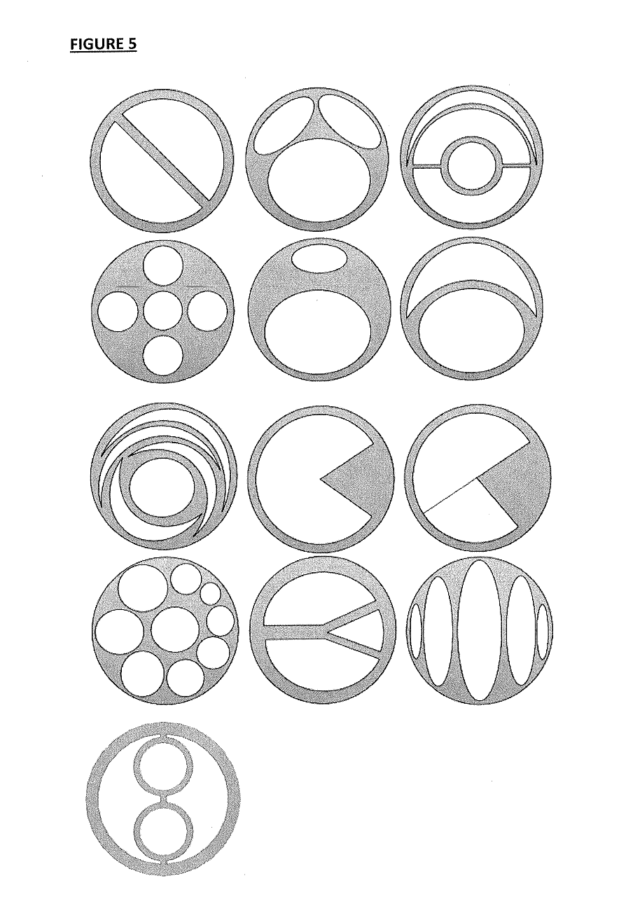

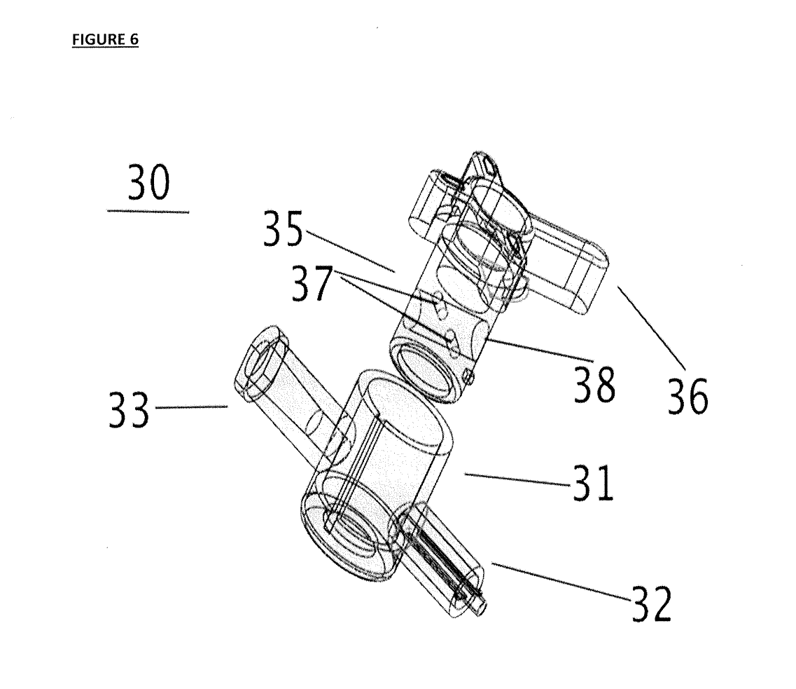

As seen in FIGS. 4A and 4B, the elongate shaft 20 (shown in cross-section) may comprise a multi-lumen elongate shaft 20, where the outlet ports 21 of the elongate shaft 20 outer surface are fluidly connected to a radial delivery lumen 25 of the elongate shaft 20, while the distal outlet 28 (not shown) is fluidly connected to a separate distal delivery lumen 26 of the elongate shaft 20.

The number, spacing, shape and arrangement of the outlet ports 21 may be varied depending on the desired characteristics of the radial delivery spray pattern (e.g. creating a uniform delivery spray pattern along the length of the elongate member 20). In some embodiments, outlet ports 21 may be provided along the entire length of the elongate shaft 20. In other embodiments, outlet ports 21 may be limited to an infusion zone on the elongate shaft 20. In such embodiments, the infusion zone may include markers (e.g. radiopaque marker bands) at proximal and/or distal ends of the infusion zone to assist in placing the infusion zone in the proper location.

With respect to shape, the shape of the outlet ports 21 may comprise any number of geometrical configurations. For example, the outlet ports 21 may be round, oblong, triangular, rectangular, comprise slits, comprise micronozzles, etc. The cross-section of the individual outlet ports 21 may be constant across the width the wall of the elongate shaft 20. Alternatively, the cross-section of the individual outlet ports 21 may vary, e.g. the cross-section of an individual outlet port 21 on the inner surface of the elongate shaft may be larger or smaller than the cross-section of the individual outlet port 21 on the outer surface of the elongate shaft. Additionally, the outlet ports 21 may extend through the width of the wall of the elongate shaft 20 at an angle so as to define angled outlet ports 21. In certain embodiments, the cross-sections of the outlet ports 21 may all be uniform. In other embodiments, the elongate shaft 20 may be covered in outlet ports 21 having varying shapes. The size of the outlet ports 21 may be varied or the size of the outlet ports 21 may be uniform. In some embodiments, the size and shape of the outlet ports 21 may be varied at different locations on the elongate shaft 20. For example, the size of the outlet ports 21 may increase or decrease along the length of the elongate shaft 20 (e.g., so as to compensate for the loss of fluid pressure). The size of the outlet ports 21 may also decrease or increase along one side of the longitudinal axis of the elongate shaft 20. In yet another embodiment, the size and shape of the outlet ports 21 may be randomly varied along the length of the elongate shaft 20.

The geometric arrangement of the outlet ports 21 along the length of the elongate shaft 20 may also be varied or uniform. For example, the outlet ports 21 may be arranged linearly, helically, randomly, or in any other arrangement or combination of arrangements. Furthermore, the spacing between outlet ports 21 may be equidistant, or may be varied. For example, the spacing between outlet ports 21 may either be increased or decreased along the length of the elongate shaft 20. Additionally, the arrangement of the outlet ports 21 may be based on the arrangement of individual outlet ports 21, or the arrangement of the outlet ports 21 may be based on the arrangement of groups of outlet ports 21. The outlet ports 21 may also be arranged along only one side of the longitudinal axis of the elongate shaft 20, or the outlet ports 21 may be arranged around the entire circumference of the elongate shaft 20. Alternatively, the outlet ports 21 may generally be arranged around the entire circumference of the elongate shaft 20, with certain predetermined portions along the longitudinal axis of the elongate shaft 20 being free of any outlet ports 21.

The configuration of the distal outlet 28 may also be varied in order to achieve a desired distal delivery spray pattern. In an embodiment, the distal end of the elongate shaft 20 may initially be constructed so as to comprise an entirely closed distal end. Subsequently, one or more holes may then be provided at the closed distal end of the elongate shaft 20 so as to create a distal outlet 28 for the distal delivery lumen 26 of the elongate shaft 20. Similarly as described with reference to the outlet ports 21 above, the number, spacing, shape and arrangement of the holes forming the distal outlet 28 may be varied depending on the desired characteristics of the distal delivery spray pattern. For example, the cross-section of the one or more holes may be varied, and the holes may also be positioned on the closed distal end of the elongate shaft 20 at angles.

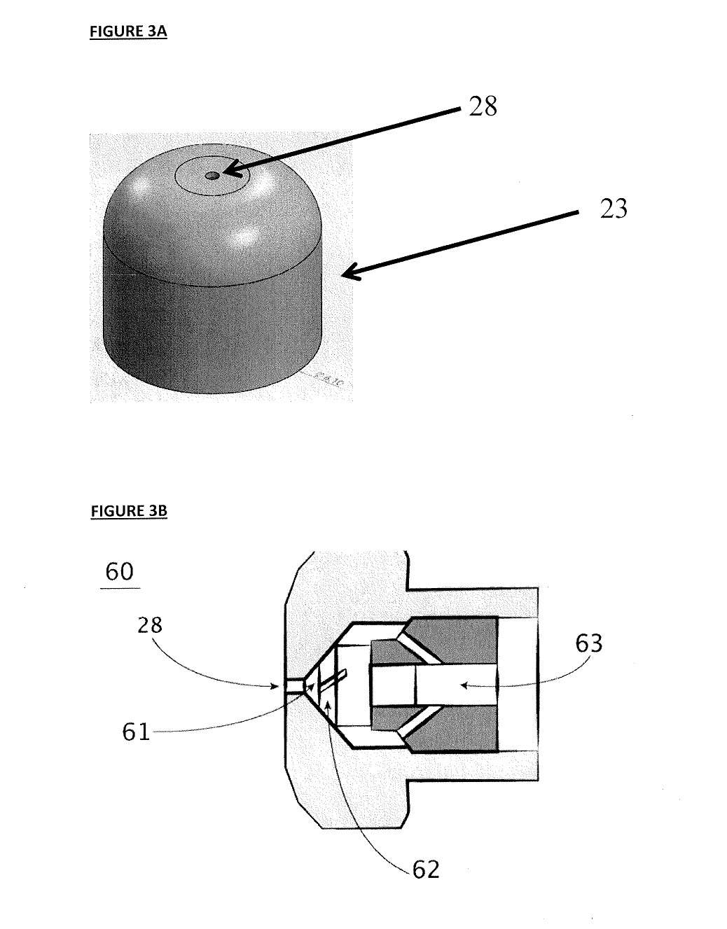

As seen in FIGS. 2 and 3A, in an embodiment, the elongate shaft 20 is not constructed with a closed distal end. Instead, a cap 23 may be provided that is attached (by any desired mechanism such as adhesive, weld, pressure, etc.) to the elongate shaft 20 distal end so as to close off the elongate shaft 20 distal end. Provided in the cap 23 can be one or more holes in fluid communication with the elongate shaft distal delivery lumen 26, wherein these one or more holes define the distal outlet 28. As with the previous embodiment, the number, spacing, shape and arrangement of holes on the cap 23 may be varied in order to a achieve a desired distal delivery spray pattern.

In an embodiment, the distal end of the radial delivery lumen 25 of the elongate shaft 20 may be constructed so as to define a closed distal end, whereas the distal delivery lumen 26 of the elongate shaft 20 may be constructed so as to define an open distal end. In this embodiment, a nozzle 60 (e.g. a nozzle such as the nozzle 60 illustrated in FIG. 3B) may be attached or inserted into the open distal end of the distal delivery lumen 26 to define a distal outlet 28. The design of the nozzle 60 may be chosen from any number of designs, depending on the desired distal delivery spray pattern to be achieved. As seen in FIG. 3B, in one embodiment, the nozzle 60 comprises a distal outlet 28, a swirl chamber 61, a swirl core 62, and an inlet 63.

As described above, the elongate shaft 20 is a multilumen elongate shaft 20 having a radial delivery lumen 25 and distal delivery lumen 26. In an embodiment, as illustrated in FIGS. 4A and 4B, the radial delivery lumen 25 and distal delivery lumen 26 are located concentrically along the length of the elongate shaft 20. Nonlimiting examples of suitable cross-sections for multiple lumens of the elongate shaft 20 are shown in FIG. 5. The arrangement of the outlet ports 21 along the elongate shaft length 20 and the arrangement of the distal outlet 28 may take on any number or arrangements depending on the type of cross-section to be employed in the design of the multi-lumen elongate shaft 20.

As shown in FIG. 2, in an embodiment, the medicament source comprises a syringe 50.

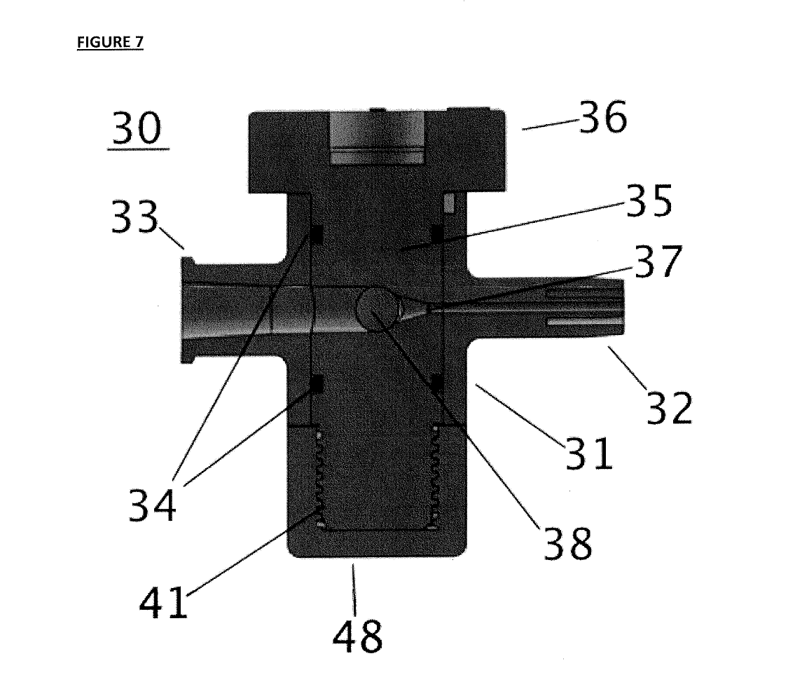

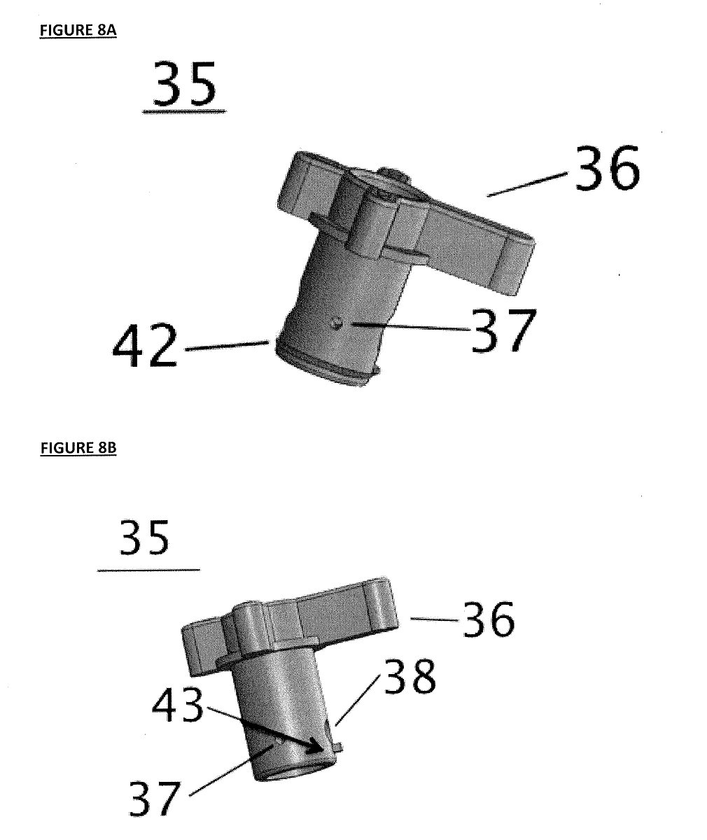

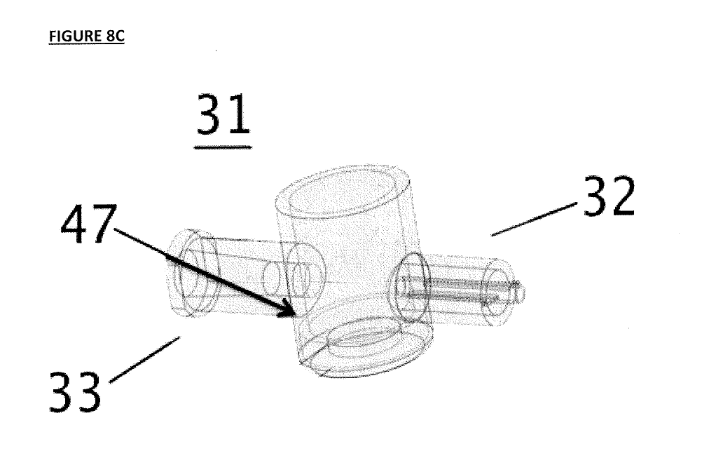

The mechanism controls the delivery spray pattern of the elongate shaft 20 in a predetermined manner so as to achieve desired radial and/or distal delivery spray patterns. As seen in FIGS. 1A, 1B, and 2, in an embodiment, the mechanism for controlling the delivery spray pattern of the elongate shaft 20 comprises a stopcock 30. The stopcock 30 comprises a bore 35 and a casing 31. As seen in FIGS. 7 and 8A-C, the bore 35 and casing 31 may be operatively connected via various ways of connecting such stopcock components. As seen in FIG. 7, in one embodiment the bore 35 and casing 31 are connected via a connection between a threaded surface 41 of the bore 35 and a nut 48. Alternatively, as seen in FIGS. 8A and 8C, the bore 35 and casing 31 are connected via a connection between a ridge 42 on the bore 35 and a track 47 on the casing 31. In yet another embodiment, as seen in FIG. 8B, the bore 35 and casing 31 are connected via a connection between a pillar 43 on the bore 35 and a track on the casing 31.

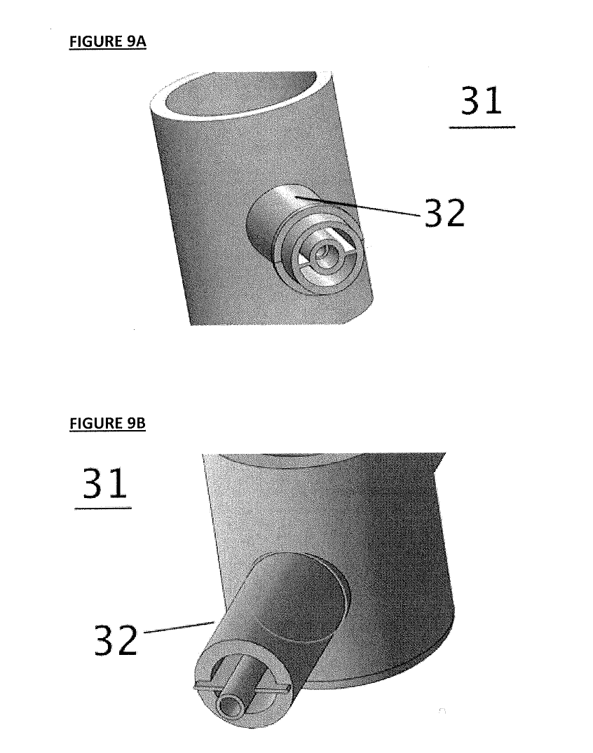

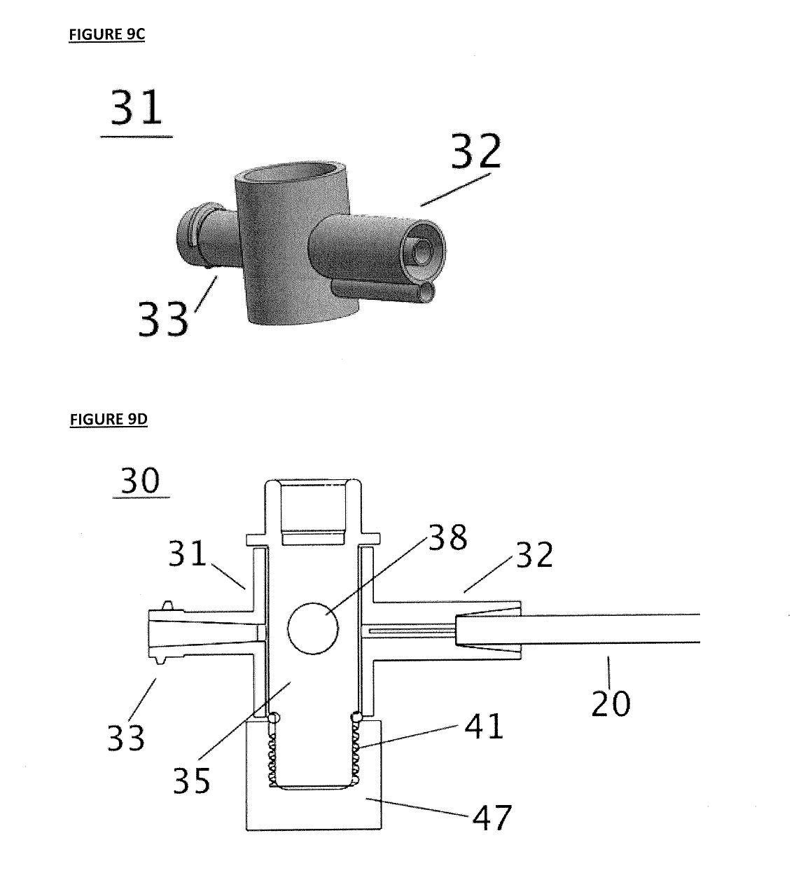

The casing 31 comprises an inlet stem 33 comprising an attachment (e.g. a luer lock or any other connection) used to connect the stopcock 30 to the medicament source. The casing 31 further comprises an outlet stem 32 comprising an attachment for connecting the stopcock 30 to the elongate shaft 20. In an embodiment, elongate shaft 20 is attached to the outlet stem 32 by positioning the elongate shaft 20 over the outlet stem 32. In another embodiment, such as the one seen in FIG. 9D, the elongate shaft 20 is attached to the outlet stem 32 by positioning the elongate shaft 20 inside the outlet stem 32. As seen in FIGS. 9A-9C, the outlet stem 32 may be constructed so as to have a cross-section that mirrors the cross-section of the elongate tube 20.

As seen in FIGS. 6 and 7, bore 35 is shaped so as to fit inside the casing 31 of the stopcock 30. At its top end, the bore 35 has a handle 36. In an embodiment, bore 35 and casing 31 are manufactured as one unit so as to minimize the leakage of any medicament flowing through the stopcock 30. Alternatively or additionally, the bore 35 may comprise one or more O-rings 34 to prevent any medicament flowing from the inlet stem 33 of the casing 31 to the outlet stem 32 of the casing 31 from seeping out of the stopcock 30.

As seen in FIGS. 6 and 7, the bore 35 is provided with two or more channels 37, 38. The two or more channels 37, 38 may have both varied cross-sections and sizes. The cross-sections and sizes of the channels 37, 38 correspond to the cross-sections and sizes of the distal delivery lumen 26 and radial delivery lumen 25 (respectively) of the elongate shaft 20. Thus, by rotating the stopcock 30, a user may control medicament flow to both the radial delivery lumen 25 and distal delivery lumen 26 simultaneously, or limit medicament flow to only the distal delivery lumen 26. In such a manner, the user can select between providing both a radial and distal delivery spray pattern, or can limit the delivery spray pattern to only a distal delivery spray pattern.

In an embodiment, the stopcock includes the casing and the bore is rotatable in the casing. The casing includes the inlet stem which is attached to the medicament source. The outlet stem is attached to the proximate end of the elongate shaft. The bore includes a first channel and a second channel. The first channel and the second channel each extend through the body of the bore. The first channel has a cross-section that matches the cross-section of the radial delivery lumen and the distal delivery lumen. In other words, the first channel has a cross-section that corresponds to the size and the shape of the combined cross-section of the radial delivery lumen and the distal delivery lumen. The second channel has a cross-section that matches the cross-section (matches the size and the shape) of the distal delivery lumen.

Rotation of the bore to a first position places the first channel in fluid communication with (i) the radial delivery lumen and the distal delivery lumen (on the outlet side of the stopcock) and (ii) the medicament source (on the inlet side of the stopcock). When the bore is in the first position, medicament flows from the medicament source, through the first channel, into the radial delivery lumen, out of the radial ports, into the distal delivery lumen, and out of the distal outlet. When the bore is in the first position, the elongate shaft delivers both a radial delivery spray pattern (through the ports) of medicament and also delivers a distal delivery spray pattern (through the distal outlet) of medicament.

The second channel has a cross-section that matches the cross-sectional size and shape of the distal delivery lumen. Rotation of the bore to a second position places the second channel in fluid communication with (i) the distal delivery lumen and (ii) the medicament source. When the bore is in the second position, medicament flows from the medicament source, through the second channel, into the distal delivery lumen, out of the distal outlet. When the bore is in the second position, the elongate shaft delivers a distal delivery spray pattern (through the ports) of medicament. When the bore is in the second position, a portion of the bore (i.e., a portion of the bore body) blocks fluid communication between the radial delivery lumen and the medicament source. When the bore is in the second position, no medicament is delivered from the radial ports.

In an embodiment, the bore includes a third channel. The third channel has a cross-section that matches the cross-section of the radial delivery lumen. Rotation of the bore to a third position places the third channel in fluid communication with (i) the radial delivery lumen and (ii) the medicament source. When the bore is in the third position, medicament flows from the medicament source, through the third channel, into the radial delivery lumen, and out of the radial ports. When the bore is in the third position, the elongate shaft delivers a radial delivery spray pattern (through the radial ports) of medicament. When the bore is in the third position, a portion of the bore (i.e., a portion of the bore body) blocks fluid communication between the distal delivery lumen and the medicament source. When the bore is in the third position, no medicament is delivered from the distal outlet.

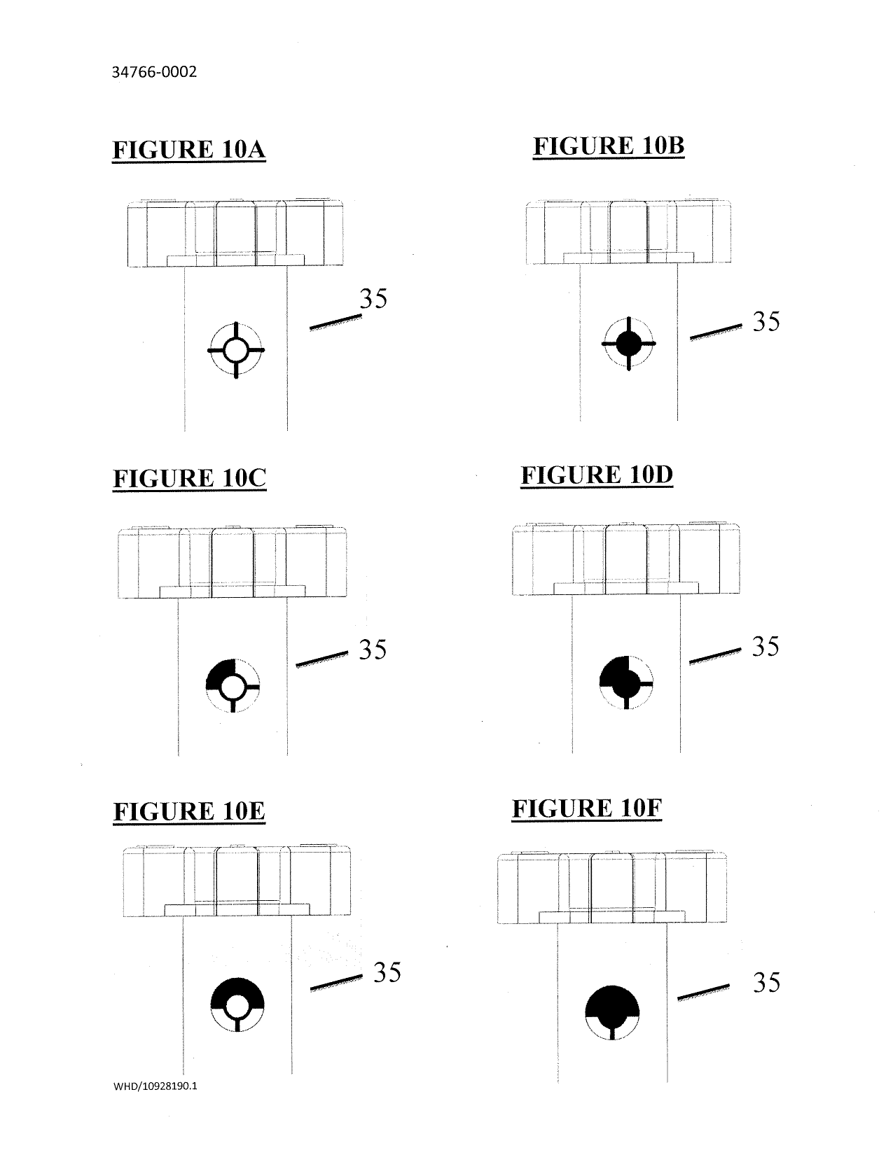

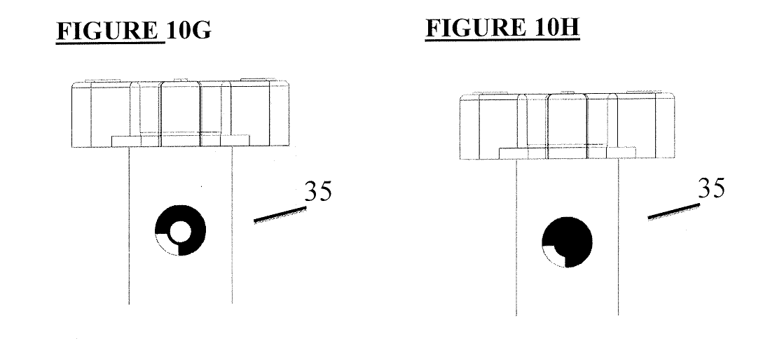

In certain embodiments, it may be desired to further limit the delivery spray pattern to only a radial delivery spray pattern. When limiting the delivery spray pattern to a radial only delivery, or alternately when providing both a combined radial and distal delivery spray pattern, a user may wish to limit the radial delivery spray pattern to only one side, or only along a specific portion of the circumference of the elongate shaft 20 (e.g. bottom half, upper half, right side, left side, etc. of the longitudinal axis of the elongate shaft 20). To that effect, the bore 35 of the stopcock 30 may be provided with additional channels to selectively block medicament flow to portions of the radial delivery lumen. As seen in FIGS. 10A-H, bore 35 may include additional channels that generally match the cross-section and size of the radial delivery lumen 25. As seen in FIGS. 10A-H, a portion of these additional channels is blocked. By selectively blocking portions of these additional channels, any number of desired radial or combination radial and distal delivery spray patterns may be achieved.

In an embodiment, a bore 35 comprising a channel of FIG. 10A would provide for both distal delivery and fully circumferential radial delivery. FIG. 10A shows a nonlimiting example of the cross-section shape and size of the first channel when the bore 35 is rotated to the first position.

In an embodiment, a bore 35 comprising a channel of FIG. 10B would provide only for fully circumferential radial delivery. FIG. 10B shows a nonlimiting example of the cross-section size and shape of the second channel when the bore 35 is rotated to the third position.

In an embodiment, a bore 35 with a channel configuration opposite to the channel configuration shown in FIG. 10B would include structure blocking the outer ring—structure blocking the channel ring portion that is open in FIG. 10B. The bore 35 with channel configuration opposite to the bore of FIG. 10B would have a channel that is open in the middle channel circle of FIG. 10B—an open channel where the blocked circle is shown in FIG. 10B. The bore with channel configuration opposite to the bore 35 of FIG. 10B is a nonlimiting example of the cross-section size and shape of the second channel when the bore is rotated to the second position.

In an embodiment, a bore 35 comprising a channel of FIG. 10C would provide for both distal and ¾ circumferential radial delivery, wherein radial delivery along the top left quadrant of the elongate shaft 20 would be blocked (bore 35 rotated to a fourth position).

In an embodiment, a bore 35 comprising a channel of FIG. 10D would provide only for ¾ circumferential radial delivery, wherein radial delivery along the top left quadrant of the elongate shaft 20 would be blocked (bore 35 rotated to a fifth position).

In an embodiment, a bore 35 comprising a channel of FIG. 10E would provide for both distal and ½ circumferential radial delivery, wherein radial delivery along the top half of the elongate shaft 20 would be blocked (bore 35 rotated to a sixth position).

In an embodiment, a bore 35 comprising a channel of FIG. 10F would provide only for ½ circumferential radial delivery, wherein radial delivery along the top half of the elongate shaft 20 would be blocked (bore 35 rotated to a seventh position).

In an embodiment, a bore 35 comprising a channel of FIG. 10G would provide for both distal and ¼ circumferential radial delivery, wherein radial delivery would be limited to only the length of the bottom left quadrant of the elongate shaft 20 (bore 35 rotated to an eighth position).

In an embodiment, a bore 35 comprising a channel of FIG. 10H would provide for only ¼ circumferential radial delivery, wherein radial delivery would be limited to only the length of the bottom left quadrant of the elongate shaft 20 (bore 35 rotated to a ninth position). It should be understood that any number of channels as described with reference to FIGS. 10A-H, or any number of channels having blocked configurations other than those explicitly described can be provided in bore 35 so as to provide a user with the desired amount of selectivity to achieve various delivery spray patterns.

In an embodiment, the handle 36 of the bore 35 is provided with indications of the configurations of the channels so as to inform a user of the type of delivery spray pattern that will be produced.

In an embodiment, the medicament delivery system 10 (not shown), both the radial outlet ports 21 and distal outlet 28 are fluidly connected to a single delivery lumen on the elongate shaft 20. In this embodiment, the mechanism for controlling the delivery spray pattern of the elongate shaft 20 comprises a cover surrounding the elongate shaft 20. The cover comprises a plurality of openings that generally mirror the arrangement of the elongate shaft 20 outlet ports 21 and distal outlet 28. The cover is rotatably connected to the proximal end of the elongate shaft 20 at its proximal end. In use, when a combined distal and radial delivery spray pattern is desired, the cover is rotated such that the plurality of openings on the cover are aligned with the outlet ports 21 of the elongate shaft 20. Alternatively, when a distal only delivery spray pattern is desired, the cover is rotated such that elongate shaft 20 outlet ports 21 and the plurality of openings on the cover are brought out of alignment, with the cover thus blocking medicament from flowing out of the elongate shaft 20 outlet ports 21 in a radial delivery spray pattern.

In an alternative embodiment of the medicament delivery system 10 (not shown), both the radial outlet ports 21 and distal outlet 28 are fluidly connected to a single delivery lumen on the elongate shaft 20. In this embodiment, the mechanism for controlling the delivery spray pattern of the elongate shaft 20 comprises a seal member that surrounds the elongate member 20. Seal member is provided with only a distal opening aligned with the distal outlet 28 of the elongate shaft 20. Provided at locations along the elongate shaft 20 between the seal member and the elongate member 20 outlet ports 21 are a plurality of valve members initially blocking the flow of medicament through the outlet ports 21, thereby initially restricting the medicament delivery system 10 to only a distal delivery spray pattern. When a combination radial delivery spray pattern and distal delivery spray pattern is desired, the valve members may be actuated to allow medicament to flow out through the outlet ports 21 and past the seal member so as to provide a combination radial and distal delivery spray pattern. For example, the valve members may comprise a plurality of puncturing members, and actuation may comprise the step of pinching the seal member against the elongate shaft 20 so as to allow the puncturing member to create outlets along the seal member through which medicament flowing through the outlet ports 21 can flow so as to generate a radial delivery spray pattern.

In an embodiment, the elongate shaft 20 is provided with features for increasing its steerability. Any number of features can be used to provide the elongate shaft with the desired level of pushability, torqueability and flexibility. As seen in FIG. 4B, in an embodiment, the elongate shaft 20 is constructed with a wire lumen 27 extending longitudinally along one side of the elongate shaft 20. A mandrel or stylet is positioned within the wire lumen 27 so as to provide the elongate shaft 20 with a desired steerability. In one embodiment, the mandrel or stylet is removably positioned within the wire lumen 27. In other embodiments, the mandrel or stylet may be manufactured integrally with the elongate shaft 20. The mandrel or stylet can comprise a uniform circumference along the entirety of its length, or alternatively can be tapered. In another embodiment, the wire lumen 27 may be sized to accommodate a guidewire over which the elongate shaft 20 may be inserted into a patient.

Alternatively, or in addition to the use of a wire lumen 27 and mandrel or stylet, and/or guidewire, the elongate shaft 20 itself may be constructed in a manner to provide it with the desired pushability, torqueability, and flexibility. In one embodiment, the elongate shaft 20 may be composed of in part, or in whole, of a hypotube surrounded (if so desired) partially or entirely by a polymer jacket. Alternatively, or in addition to a hypotube, the elongate shaft 20 may be constructed so as to include braided, coiled, and/or woven materials. In any of these embodiments, multiple transition zones may be provided along the length of the elongate shaft 20 to achieve the desired steerability. Additionally, in such embodiments the hypotube, braid, woven material, and/or coil may extend along the entirety or along only a portion of the length of the elongate shaft 20. Furthermore, the hypotube, braid, woven material, and/or coil may extend only along the entire circumference of the elongate shaft 20 or along only one side of the elongate shaft 20.

Additionally or alternatively, desired steerability may be achieved by varying the materials from which the elongate shaft 20 itself is constructed. Materials having appropriate malleability and durometer are selected to construct an elongate shaft 20 having desired steerability characteristics. The elongate shaft 20 steerability may also be increased by, for example, constructing different portions of the elongate shaft 20 from different materials, providing transitions zones along the length of the elongate shaft 20, and/or scoring or providing notches in the elongate shaft 20, etc.

Thus, any number of mechanisms can be employed in order to position the elongate shaft 20 in a desired location within a patient. In addition to or as an alternative to any of the options described above, the elongate shaft 20 may be introduced into a patient via a sheath. Also, the elongate shaft 20 may comprise a weighted distal tip. Furthermore, as seen in FIG. 2 the elongate shaft 20 may comprise a curved distal tip. The incorporation of any of the above described ways for increasing steerability (or any other means of increasing steerability as known in the art) will allow a user to bend the distal tip of an initially straight elongate shaft 20 to any desired angled prior to use of the medicament delivery system 10. Alternatively, the elongate shaft 20 may be constructed with a preformed distal curve. The angle of the preformed distal curve can be any angle as would be desired by a user, such as, e.g. an angle of approximately 60 degrees.

In an embodiment, the elongate shaft 20 is constructed with a preformed distal curve, the preformed distal curve may be the only way of increasing the steerability of the elongate shaft 20. Alternatively, the preformed distal curve may be employed in addition to any of the other aforementioned ways of increasing steerability of the elongate shaft 20. Therefore, a user has the option of altering the angle of preformed distal tip during use.

The medicament delivery system 10 of the present disclosure may be used in any number of known treatment methods of delivering medicament to a patient (e.g. transesophageal, transoral, nasal, aural, vascular, oral, vaginal, rectal, superficial, etc.). The medicament delivery system 10 of the present disclosure can be used to deliver any number of or any combination of desired medicaments. During use of the medicament delivery system 10, a single medicament or combination of medicaments can be delivered. Alternatively, once the elongate shaft 20 has been positioned at a desired location in a patient, a user may subsequently use the medicament delivery system 10 to deliver any number or medicaments or combination of medicaments as desired.

The term “medicament” as used herein, includes any feasible drugs, prodrugs, proteins, gene therapy preparations, cells, diagnostic agents, contrast or imaging agents, biologicals, water, saline, etc. Additionally, in some embodiments (e.g., where the medicament delivery system 10 of the present disclosure is used for research purposes) the medicament may also comprise substances such as carcinogens and toxins. Such substances may be in bound or free form, liquid or solid, colloid or other suspension, solution or may be in the form of a gas or other fluid or non-fluid.

In an embodiment, it may be desirable to deliver a topical anesthetic via the medicament delivery system 10 of the present disclosure. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

Additionally or alternatively, in applications where it is desired to treat or prevent a microbial infection, the substance delivered may comprise pharmaceutically acceptable salt or dosage form of an antimicrobial agent (e.g., antibiotic, antiviral, antiparacytic, antifungal, etc.), a corticosteroid or other anti-inflammatory (e.g., an NSAID), a decongestant (e.g., vasoconstrictor), a mucous thinning agent (e.g., an expectorant or mucolytic), an agent that prevents of modifies an allergic response (e.g., an antihistamine, cytokine inhibitor, leucotriene inhibitor, IgE inhibitor, immunomodulator), etc. Some nonlimiting examples of antimicrobial agents that may be used in this disclosure include acyclovir, amantadine, aminoglycosides (e.g., amikacin, gentamicin and tobramycin), amoxicillin, amoxicillin/clavulanate, amphotericin B, ampicillin, ampicillin/sulbactam, atovaquone, azithromycin, cefazolin, cefepime, cefotaxime, cefotetan, cefpodoxime, ceftazidime, ceftizoxime, ceftriaxone, cefuroxime, cefuroxime axetil, cephalexin, chloramphenicol, clotrimazole, ciprofloxacin, clarithromycin, clindamycin, dapsone, dicloxacillin, doxycycline, erythromycin, fluconazole, foscarnet, ganciclovir, atifloxacin, imipenem/cilastatin, isoniazid, itraconazole, ketoconazole, metronidazole, nafcillin, nafcillin, nystatin, penicillin, penicillin G, pentamidine, piperacillin/tazobactam, rifampin, quinupristin-dalfopristin, ticarcillin/clavulanate, trimethoprim/sulfamethoxazole, valacyclovir, vancomycin, mafenide, silver sulfadiazine, mupirocin (e.g., Bactroban Nasal®, Glaxo SmithKline, Research Triangle Park, N.C.), nystatin, triamcinolone/nystatin, clotrimazole/betamethasone, clotrimazole, ketoconazole, butoconazole, miconazole, tioconazole, detergent-like chemicals that disrupt or disable microbes (e.g., nonoxynol-9, octoxynol-9, benzalkonium chloride, menfegol, and N-docasanol); chemicals that block microbial attachment to target cells and/or inhibits entry of infectious pathogens (e.g., sulphated and sulponated polymers such as PC-515 (carrageenan), Pro-2000, and Dextrin 2 Sulphate); antiretroviral agents (e.g., PMPA gel) that prevent retroviruses from replicating in the cells; genetically engineered or naturally occurring antibodies that combat pathogens such as anti-viral antibodies genetically engineered from plants known as “plantibodies;” agents which change the condition of the tissue to make it hostile to the pathogen (such as substances which alter mucosal pH (e.g., Buffer Gel and Acidform); non-pathogenic or “friendly” microbes that cause the production of hydrogen peroxide or other substances that kill or inhibit the growth of pathogenic microbes (e.g., lactobacillus); or antimicrobial proteins or peptides.

Additionally or alternatively, in some applications where it is desired to treat or prevent inflammation the substances delivered in this disclosure may include various steroids or other anti-inflammatory agents (e.g., nonsteroidal anti-inflammatory agents or NSAIDS), analgesic agents or antipyretic agents. For example, corticosteroids, such as beclomethasone (Vancenase® or Beconase®, flunisolide)(Nasalide®, fluticasone proprionate)(Flonase®, triamcinolone acetonide)(Nasacort®, budesonide (Rhinocort Aqua®, loterednol etabonate (Locort) and mometasone (Nasonex®) may be used. Other salt forms of the aforementioned corticosteroids may also be used. Also, other non-limiting examples of steroids that may be useable in the present disclosure include but are not limited to aclometasone, desonide, hydrocortisone, betamethasone, clocortolone, desoximetasone, fluocinolone, flurandrenolide, mometasone, prednicarbate; amcinonide, desoximetasone, diflorasone, fluocinolone, fluocinonide, halcinonide, clobetasol, augmented betamethasone, diflorasone, halobetasol, prednisone, dexamethasone and methylprednisolone. Other anti-inflammatory, analgesic or antipyretic agents that may be used include the nonselective COX inhibitors (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam) and alkanones such as nabumetone) and Selective COX-2 Inhibitors (e.g., diaryl-substituted furanones such as rofecoxib; diaryl-substituted pyrazoles such as celecoxib; indole acetic acids such as etodolac and sulfonanilides such as nimesulide).

Additionally or alternatively, in some applications, such as those where it is desired to treat or prevent an allergic or immune response and/or cellular proliferation, the substances delivered in this disclosure may include a) various cytokine inhibitors such as humanized anti-cytokine antibodies, anti-cytokine receptor antibodies, recombinant (new cell resulting from genetic recombination) antagonists, or soluble receptors; b) various leukotriene modifiers such as zafirlukast, montelukast and zileuton; c) immunoglobulin E (IgE) inhibitors such as Omalizumab (an anti-IgE monoclonal antibody formerly called rhu Mab-E25) and secretory leukocyte protease inhibitor) and d) SYK Kinase inhibitors such as an agent designated as “R-112” manufactured by Rigel Pharmaceuticals, Inc, or South San Francisco, Calif.

Additionally or alternatively, in some applications, such as those where it is desired to shrink mucosal tissue, cause decongestion or effect hemostasis, the substances delivered in this disclosure may include various vasoconstrictors for decongestant and or hemostatic purposes including but not limited to pseudoephedrine, xylometazoline, oxymetazoline, phenylephrine, epinephrine, etc.

Additionally or alternatively, in some applications, such as those where it is desired to facilitate the flow of mucous, the substances delivered in this disclosure may include various mucolytics or other agents that modify the viscosity or consistency of mucous or mucoid secretions, including but not limited to acetylcysteine (Mucomyst™, Mucosil™) and guaifenesin. In one particular embodiment, the substance delivered by this disclosure comprises a combination of an anti-inflammatory agent (e.g. a steroid or an NSAID) and a mucolytic agent.

Additionally or alternatively, in some applications such as those where it is desired to prevent or deter histamine release, the substances delivered in this disclosure may include various mast cell stabilizers or drugs which prevent the release of histamine such as cromolyn (e.g., Nasal Chrom®) and nedocromil. Additionally or alternatively, in some applications such as those where it is desired to prevent or inhibit the effect of histamine, the substances delivered in this disclosure may include various antihistamines such as azelastine (e.g., Astylin®), diphenhydramine, loratidine, etc.

Additionally or alternatively, in some applications, the substances delivered in this disclosure may include other classes of substances that are used to treat rhinitis, nasal polyps, nasal inflammation, and other disorders of the ear, nose and throat including but not limited to anti-cholinergic agents that tend to dry up nasal secretions such as ipratropium (Atrovent Nasal®), as well as other agents not listed here.

Additionally or alternatively, in some applications such as those where it is desired to draw fluid from polyps or edematous tissue, the substances delivered in this disclosure may include locally or topically acting diuretics such as furosemide and/or hyperosmolar agents such as sodium chloride gel or other salt preparations that draw water from tissue or substances that directly or indirectly change the osmolar content of the mucous to cause more water to exit the tissue to shrink the polyps directly at their site.

Additionally or alternatively, in some applications such as those wherein it is desired to treat a tumor or cancerous lesion, the substances delivered in this disclosure may include antitumor agents (e.g., cancer chemotherapeutic agents, biological response modifiers, vascularization inhibitors, hormone receptor blockers, cryotherapeutic agents or other agents that destroy or inhibit neoplasia or tumorigenesis) such as; alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6 mercaptopurine and 5-fluorouracil (5FU), antitumor antibiotics and other compounds that act by binding or intercalating DNA and preventing RNA synthesis (e.g., doxorubicin, daunorubicin, epirubicin, idarubicin, mitomycin-C and bleomycin) plant (vinca) alkaloids and other anti-tumor agents derived from plants (e.g., vincristine and vinblastine), steroid hormones, hormone inhibitors, hormone receptor antagonists and other agents which affect the growth of hormone-responsive cancers (e.g., tamoxifen, herceptin, aromatase ingibitors such as aminoglutethamide and formestane, trriazole inhibitors such as letrozole and anastrazole, steroidal inhibitors such as exemestane), antiangiogenic proteins, small molecules, gene therapies and/or other agents that inhibit angiogenesis or vascularization of tumors (e.g., meth-1, meth-2, thalidomide), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAI), combretastatin A4 prodrug (CA4P), SU6668, SU11248, BMS-275291, COL-3, EMD 121974, IMC-1C11, IM862, TNP-470, celecoxib (Celebrex), rofecoxib (Vioxx), interferon alpha, interleukin-12 (IL-12), biological response modifiers (e.g., interferon, bacillus calmette-guerin (BCG), monoclonal antibodies, interluken 2, granulocyte colony stimulating factor (GCSF), etc.), PGDF receptor antagonists, herceptin, asparaginase, busulphan, carboplatin, cisplatin, carmustine, cchlorambucil, cytarabine, dacarbazine, etoposide, flucarbazine, flurouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxol, taxotere, analogs/congeners and derivatives of such compounds as well as other antitumor agents not listed here.

Additionally or alternatively, in some applications such as those where it is desired to grow new cells or to modify existing cells, the substances delivered in this disclosure may include cells (mucosal cells, fibroblasts, stem cells or genetically engineered cells) as well as genes and gene delivery vehicles like plasmids, adenoviral vectors or naked DNA, mRNA, etc. injected with genes that code for anti-inflammatory substances, etc., and, as mentioned above, osteoclasts that modify or soften bone when so desired, cells that participate in or effect mucogenesis or ciliagenesis, etc.

In an embodiment of the present disclosure, the medicament delivery system 10 is an endotracheal applicator for the delivery of a topical anesthetic to the airway during laryngoscopy. The term “airway,” as used herein, includes the oropharynx (the mouth and throat), nasal cavities, nasopharynx (the nose and throat convergence), hypopharynx (the deep throat), larynx (the voice box, including false vocal cords and true vocal cords), glottis, proximal and distal trachea (the windpipe), bronchi including primary, secondary, and tertiary bronchi (the smaller breathing tubes), and any combination thereof. Exemplary methods of using the device will now be disclosed.

In an embodiment, a direct laryngoscope blade, such as a Miller (straight) blade or Macintosh (curved) blade, is inserted into a patient's mouth and positioned in the deep throat to provide direct line-of-sight visualization of the patient's glottic opening (windpipe opening) and vocal cords. With the elongate shaft 20 in a generally straight configuration, the elongate shaft 20 of the medicament delivery system 10 is advanced under direct line-of-sight to the patient's glottic opening and the tip of the elongate shaft 20 is positioned past the patient's vocal cords in the patient's trachea (windpipe). Once the elongate shaft 20 is in place, topical anesthetic solution is applied to the patient's glottis, vocal cords, and trachea by selecting a radial, distal, or combination of the radial spray pattern and distal delivery spray pattern.

In an embodiment, the topical anesthetic solution is applied using a combination of both the radial delivery spray pattern and the distal delivery spray pattern. After the topical anesthetic solution is applied to the laryngotrachea, the elongate shaft 20 is removed from the patient. Following this, the operator will typically continue to maintain direct line-of-sight visualization of the patient's glottic opening and proceed with endotracheal (“tracheal”) tube placement.

In an embodiment, an indirect, non-channeled laryngoscope blade, such as a Glidescope or Karl Stortz D-blade, is inserted into the patient's mouth and positioned in the deep throat to provide a view of the patient's glottic opening (windpipe opening) and vocal cords on the laryngoscope monitor. With the elongate shaft 20 distal end curved at an approximately 60 degree angle (as seen in FIG. 2) the elongate shaft 20 is placed in the patient's mouth and advanced into the distal throat until visualized on the indirect laryngoscope monitor. Once visualized on the indirect laryngoscope monitor, the tip of the elongate shaft 20 is positioned past the patient's vocal cords in the patient's trachea (windpipe). With the elongate shaft 20 in this position, topical anesthetic solution is applied to the patient's glottis, vocal cords, and trachea by selecting a radial, distal, or combination radial spray pattern and distal delivery spray pattern.

In an embodiment, the topical anesthetic solution is applied using a combination of both the radial spray pattern and distal delivery spray pattern. After the topical anesthetic solution is applied to the laryngotrachea, the elongate shaft 20 is removed from the patient. Following this, the operator will typically continue to maintain indirect visualization of the patient's glottic opening on the laryngoscope monitor and proceed with tracheal tube placement.

In an embodiment, an indirect, non-channeled laryngoscope blade, such as a Glidescope or Karl Stortz D-blade, is inserted into the patient's mouth and positioned in the deep throat to provide a view of the patient's glottic opening (windpipe opening) and vocal cords on the laryngoscope monitor. With the elongate shaft 20 distal end curved at an approximately 60 degree angle (as seen in FIG. 2), the elongate shaft 20 is placed into a tracheal tube. The combination of the elongate shaft 20 and tracheal tube is then placed in the patient's mouth and advanced into the distal throat until visualized on the indirect laryngoscope monitor. Once visualized on the indirect laryngoscope monitor, the tip of the elongate shaft 20/tracheal tube combination is positioned proximal to and in-line with the patient's vocal cords. With the elongate shaft 20/tracheal tube combination in this position, topical anesthetic solution is applied to the patient's glottis, vocal cords by selecting a radial, distal, or combination radial and distal delivery spray pattern so as to reduce anesthetic loss in the tracheal tube.

In an embodiment, the topical anesthetic solution is applied using a distal only delivery spray pattern. After the topical anesthetic solution is applied to the laryngotrachea, the elongate shaft 20/tracheal tube combination is advanced such that the tracheal tube tip is positioned between the vocal cords. In this position, the elongate shaft 20 is retracted back into the tracheal tube and the tracheal tube is advanced further into the patient's trachea. Once the tracheal tube is fully seated in the trachea, the elongate shaft 20 is fully removed from the tracheal tube.

In an embodiment, an indirect, channeled laryngoscope, such as an Airtraq or Pentax Airway Scope, is prepared in accordance with the manufacturer's instructions, which typically includes preloading a tracheal tube in the laryngoscope's channel. After preloading the tracheal tube in the laryngoscope's channel, the elongate shaft 20, is loaded in the preloaded tracheal tube. The elongate shaft 20 may be loaded in the preloaded tracheal tube after the indirect, channeled laryngoscope is positioned in the patient's airway, or alternatively, the elongate shaft 20 may be loaded in the preloaded tracheal tube prior to positioning the indirect, channeled laryngoscope in the patient's airway. The prepared indirect, channeled laryngoscope blade is inserted into the patient's mouth and positioned in the deep throat to provide a view of the patient's glottis opening (windpipe opening) and vocal cords on the laryngoscope monitor. Once visualized on the indirect laryngoscope monitor, the tip of the channeled laryngoscope device combination is positioned proximal to and in-line with the patient's vocal cords. With the channeled laryngoscope device combination in this position, topical anesthetic solution is applied to the patient's glottis, vocal cords, and trachea by selecting a radial, distal, or a combination of the radial spray pattern and the distal delivery spray pattern.

In an embodiment, the topical anesthetic solution is applied using a distal only delivery spray pattern so as to reduce anesthetic loss in the tracheal tube. After the topical anesthetic solution is applied to the laryngotrachea, the elongate shaft 20 is removed from the tracheal tube and the patient. Following this, the operator will typically continue to maintain indirect visualization of the patient's glottic opening on the indirect laryngoscope monitor and proceed with tracheal tube placement.

In each of the above described methods, it is to be understood that when a radial delivery spray pattern is selected, the radial delivery spray pattern may be selected to be either fully circumferential or only partially circumferential. Furthermore, in each of the above described method embodiments, placement of the laryngoscope occurs after either adequate topical anesthesia of the oropharyngeal (mouth and throat) and hypopharyngeal (deep throat above the vocal cords) areas and/or the induction of general anesthesia. Alternatively, placement of the laryngoscope may occur without adequate topical anesthesia of the oropharyngeal (mouth and throat) and hypopharyngeal (deep throat above the vocal cords) areas and/or the induction of general anesthesia. Additionally or alternatively, in each of the above methods, subsequent to placement of the tracheal tube, the elongate member 20 may be inserted through the tracheal tube and used to deliver additional topical anesthetic.

In an embodiment, the reintroduction of the elongate member 20 is done prior to removal of the tracheal tube from the patient, so as to reapply additional topical anesthetic. In this subsequent delivery, the topical anesthetic solution is applied using a distal only delivery spray pattern so as to reduce anesthetic loss in the tracheal tube.

Furthermore, in an embodiment, the medicament delivery system 10 may itself be used to provide oropharyngolaryngotracheal topical anesthetic application to a patient's oropharynx and hypopharynx prior to the induction of general anesthesia or prior to placement of the laryngoscope. In this embodiment, the elongate shaft 20 is sequentially placed in deeper portions of the patient's mouth starting with the back of the mouth and moving deeper to the back of the patient's throat. With the elongate shaft 20 in these positions, topical anesthetic solution is applied to the patient's mouth, throat, and deep throat by selecting a radial, distal, or combination radial and distal delivery spray pattern.

In an embodiment, the topical anesthetic solution is applied using a combination of both the radial and distal delivery spray patterns. After the topical anesthetic solution is applied to the oropharyngolaryngotrachea, the elongate shaft 20 is removed from the patient. Following the application of the topical anesthetic, the operator may place a direct or indirect laryngoscope and achieve visualization of the patient's glottic opening and proceed with tracheal tube placement. Alternatively, once the elongate shaft 20 is removed from the patient, the operator may proceed with any one of the above described methods of using a laryngoscope for tracheal tube placement.

It should be understood that although in each of the above described methods (as well during any other method of using the medicament delivery system 10) a user may select any number of combinations of delivery spray patterns during use of the medicament delivery system 10. For example, a user may wish to use a distal delivery spray pattern only setting during steps of the procedure where the elongate shaft 20 is located entirely within the tracheal tube, so as to reduce anesthetic loss which would otherwise occur if both a radial and distal delivery were selected. On the other hand, during steps of the procedure where the elongate shaft 20 is not positioned within a tracheal tube, or where the elongate shaft 20 extends past the tracheal tube, a user may switch to a radial only or a combined radial delivery spray pattern and distal delivery spray pattern to maximize medicament delivery to the desired treatment area. As an example, in an embodiment where the medicament delivery system 10 is used to provide the initial topical anesthetic oropharyngolaryngotracheal application to a patient's oropharynx and hypopharynx prior to the induction of general anesthesia or prior to placement of the laryngoscope, a user may initially wish to use the medicament delivery system 10 with a combined distal and radial delivery spray pattern. Subsequently, if the user introduces the elongate shaft 20 into the patient by first inserting the elongate shaft into a tracheal tube, and then inserting the combination elongate shaft 20/tracheal tube into the patient's airway simultaneously, the user may switch the means for controlling the delivery spray pattern to select a distal only delivery spray pattern of the device.

With respect to the aforementioned method embodiments, the elongate shaft 20 has a length sufficient so as to position the distal end of the elongate shaft 20 past the patient's vocal cords and into the patient's trachea. The length of the elongate shaft 20 may be varied depending on the specific requirements of the patient being treated and or the specific requirements of the method the elongate shaft 20 is to be used in. In an embodiment, the length of the elongate shaft 20 used in the aforementioned method embodiments is from 20 cm, or 25 cm to 30 cm, or 35 cm, or 40 cm.

Although the method of using the medicament delivery system 10 of the present disclosure has been described as being used with laryngoscopes during laryngoscopy, it should be understood that use of the medicament delivery system 10 is not limited to such uses for treating a patient's airway. Alternative uses of the medicament delivery system 10 include, but are not limited to: treating airway lesions, selectably delivering factors (drugs, harmful agents, cofactors, etc.) to the airway, treating vocal fold paralysis, scarring, nodules, polyps, cysts, etc.

Nonlimiting examples of further embodiments for the medicament delivery system are provided below.

In an embodiment, a medicament delivery system (E1) is provided and includes a medicament source, a flexible elongate shaft having a distal end and a proximal end. A radial delivery lumen and a distal delivery lumen extend along the length of the elongate shaft. A distal outlet is fluidly connected to the distal lumen. A plurality of ports are spaced radially along the length of the elongate shaft. The ports are fluidly connected to the radial lumen. The medicament delivery system includes a stopcock. The stopcock includes a casing and a rotatable bore in the casing. The casing includes an inlet stem attached to the medicament source and an outlet stem attached to the proximate end of the elongate shaft. The bore includes a first channel and a second channel. The first channel has a cross-section that matches the cross-section of the radial delivery lumen and the distal delivery lumen. The second channel has a cross-section that matches the cross-section of the radial delivery lumen. Rotation of the bore to a first position places the first channel in fluid communication with (i) the radial delivery lumen and the distal delivery lumen and (ii) the medicament source. In the first position, the elongate shaft delivers a radial delivery pattern and a distal delivery spray pattern. Rotation of the bore to a second position places the second channel in fluid communication with (i) the distal delivery lumen and (ii) the medicament source. In the second position, the elongate shaft delivers a distal delivery spray pattern.

In an embodiment (E2), the system of E1 includes the bore in the second position. When the bore is in the second position, a portion of the bore blocks fluid communication between the radial delivery lumen and the medicament source.

In an embodiment (E3), the system of E1 includes a bore having a third channel. The third channel has a cross-section that matches the cross-section of the radial delivery system. Rotation of the bore to a third position places the third channel in fluid communication with (i) the radial delivery lumen and (ii) the medicament source. In the third position, the elongate shaft delivers a radial delivery spray pattern.

In an embodiment (E4), the system of E3 includes the bore in the third position. When the bore is in the third position, a portion of the bore blocks fluid communication between the distal delivery lumen and the medicament source.

In an embodiment (E5), the system of E1 includes a bore that has a handle. The handle is used by a user to rotate the bore in the casing.

In an embodiment (E6), the system of E1 includes an elongate shaft that is a flexible elongate shaft.

In an embodiment (E7), the system of E6 includes a wire lumen that extends along the length of the flexible elongate shaft.

In an embodiment (E8), the system of E1 includes ports that are arranged around the entire circumference of the elongate shaft.

In an embodiment (E9) the system of E1 includes a radial delivery lumen that concentrically surrounds the distal delivery lumen.

In an embodiment (E10), the system of E1 include a cap at the distal end of the elongate shaft. The distal outlet is located in the cap.

In an embodiment (E11), the system of E1 includes a medicament source that is a syringe. The syringe contains solution of medicament.

In an embodiment (E12), the system of E11 includes a medicament that is a topical anesthetic.

In an embodiment (E13), the system of E11 includes the elongate shaft placed in a person's airway. The system delivers a spray of medicament to an organ selected from the mouth, nasal cavity, pharynx, hypopharnyx, glottis, the vocal cords, the trachea, bronchi, and combinations thereof.

The medicament delivery system (E1) may include two or more embodiments disclosed herein.

It is specifically intended that the present disclosure not be limited to the embodiments and illustrations contained herein, but include modified forms of those embodiments including portions of the embodiments and combinations of elements of different embodiments as come within the scope of the following claims.

We claim:

1. A medicament delivery system comprising:
a medicament source;
a flexible elongate shaft having a distal end and a proximal end;
a radial delivery lumen and a distal delivery lumen extending along the length of the elongate shaft;
a distal outlet fluidly connected to the distal lumen;
a plurality of ports spaced radially along the length of the elongate shaft, the ports fluidly connected to the radial lumen;
a stopcock comprising a casing and a rotatable bore in the casing, the casing comprising an inlet stem attached to the medicament source and an outlet stem attached to the proximate end of the elongate shaft;
the bore comprising a first channel and a second channel, the first channel having a cross-section that matches the cross-section of the radial delivery lumen and the distal delivery lumen, the second channel having a cross-section that matches the cross-section of the distal delivery lumen;
rotation of the bore to a first position places the first channel in fluid communication with (i) the radial delivery lumen and the distal delivery lumen and (ii) the medicament source, and the elongate shaft delivers a radial delivery pattern and a distal delivery spray pattern; and
rotation of the bore to a second position places the second channel in fluid communication with (i) the distal delivery lumen and (ii) the medicament source, and the elongate shaft delivers a distal delivery spray pattern.

2. The system of claim 1 wherein when the bore is in the second position, a portion of the bore blocks fluid communication between the radial delivery lumen and the medicament source.

3. The system of claim 1 wherein the bore comprises a third channel having a cross-section that matches the cross-section of the radial delivery system; and
rotation of the bore to a third position places the third channel in fluid communication with (i) the radial delivery lumen and (ii) the medicament source; and the elongate shaft delivers a radial delivery spray pattern.

4. The system of claim 3 wherein when the bore is in the third position, a portion of the bore blocks fluid communication between the distal delivery lumen and the medicament source.

5. The system of claim 1 wherein the bore comprises a handle for rotating the bore in the casing.

6. The system of claim 1 wherein the elongate shaft is a flexible elongate shaft.

7. The system of claim 6 comprising a wire lumen extending along the length of the flexible elongate shaft.

8. The system of claim 1 wherein the ports are arranged around the entire circumference of the elongate shaft.

9. The system of claim 1 wherein the radial delivery lumen concentrically surrounds the distal delivery lumen.

10. The system of claim 1 comprising a cap at the distal end of the elongate shaft; and the distal outlet is located in the cap.

11. The system of claim 1 herein the medicament source comprises a syringe containing a solution of medicament.

12. The system of claim 11 wherein the medicament is a topical anesthetic.

13. The system of claim 11 wherein the elongate shaft is placed in a person's airway, and the system delivers a spray of medicament to an organ selected from the group consisting of mouth, nasal cavity, pharynx, hypopharynx, glottis, vocal cords, trachea, bronchi, and combinations thereof.
